SEQUENCE LISTING (8 sequences)

SEQ ID NO: 1 - DNA, 240 bp, Artificial Sequence
  PARMS-1517 molecular marker
aatatagtcatcagtttgcacgaattctatattagattgtactttcaactcaaatattttcaggattatttgttgaattaaaacctttattttgcagattggagagaggttctgctagataccacgatggmtatggaagactgagatcatgggcatataggtcacttgaatcatacaaggtagcatggcatttggttactgtcttgtttaaagttcattcatgaaattgccatgtttcag

SEQ ID NO: 2 - DNA, 240 bp, Artificial Sequence
  PARMS-10010 molecular marker
ataaaggttccaaggaatgaagaaaagtaagtagtttatctgacgaaccatctctttaggaattttcttttctaatggaaaaactgagctcatatttttcacattagtagaagtggraatgctaacagactcagatcattaaaaagcctgccaaccaaatctgcttcagtttactctctccaggtacattcactaattacccattctttgtttcacttatccatggttagtctttattca

SEQ ID NO: 3 - DNA, 44 bp, Artificial Sequence
  Primer 1 PARMS-1517F1
gaaggtgaccaagttcatgctgttctgctagataccacgatgga

SEQ ID NO: 4 - DNA, 43 bp, Artificial Sequence
  Primer 2 PARMS-1517F2
gaaggtcggagtcaacggattttctgctagataccacgatggc

SEQ ID NO: 5 - DNA, 23 bp, Artificial Sequence
  Primer 3 PARMS-1517R
gacctatatgcccatgatctcag

SEQ ID NO: 6 - DNA, 48 bp, Artificial Sequence
  Primer 1 PARMS-10010F1
gaaggtgaccaagttcatgcttttaatgatctgagtctgttagcattt

SEQ ID NO: 7 - DNA, 48 bp, Artificial Sequence
  Primer 2 PARMS-10010F2
gaaggtcggagtcaacggatttttaatgatctgagtctgttagcattc

SEQ ID NO: 8 - DNA, 26 bp, Artificial Sequence
  Primer 3 PARMS-10010R
tctttaggaattttcttttctaatgg

(19) United States
(12) Patent Application Publication
(10) Pub. No.: US 2022/0228227 A1
(43) Pub. Date: Jul. 21, 2022

(54) Two PARMS-SNP Molecular Markers for Identifying Resistant Gene VrTAF5 of Vigna radiata (Linn.) Wilczek Cercospora Leaf Spot Disease

(71) Applicant: Jiangsu Academy of Agricultural, Nanjing City (CN)

(72) Inventors: Ranran Wu, Nanjing City (CN); Linghui Li, Ling County (CN); Xin Chen, Nanjing City (CN); Jingbin Chen, Nanjing City (CN); Yun Lin, Nanjing City (CN); Xingxing Yuan, Nanjing City (CN); Chenchen Xue, Nanjing City (CN); Qiang Yan, Nanjing City (CN)

(21) Appl. No.: 17/523,188

(22) Filed: Nov. 10, 2021

(30) Foreign Application Priority Data
Jan. 21, 2021 (CN) 2021100818284

(51) Int. Cl. C12Q 1/6895; C12Q 1/6827

(52) U.S. Cl. CPC C12Q 1/6895; C12Q 2600/156; C12Q 1/6827

(57) ABSTRACT

Two PARMS-SNP molecular markers for identifying resistant gene VrTAF5 of Vigna radiata (Linn.) Wilczek Cercospora leaf spot disease are provided, which belongs to the field of molecular genetic breeding. The two molecular markers PARMS-1517 and PARMS-10010 include SNP sites located at the 32622352 and 32613913 bases of Vigna radiata (Linn.) Wilczek chromosome 6 respectively. 240 bp sequence before and after the PARMS-1517 is shown in SEQ ID NO.1, a 131st position is SNP site, and a polymorphism is A/C; 240 bp sequence before and after the PARMS-10010 is shown in SEQ ID NO.2, a 117th position is SNP site, and a polymorphism is A/G. The PARMS-SNP molecular markers have a high specificity, accurate and reliable detection results, which can be used for rapid identifying Vigna radiata (Linn.) Wilczek Cercospora leaf spot disease resistant varieties, molecular marker-assisted selective breeding, and shortening a breeding period.

### TECHNICAL FIELD

The invention relates to the field of molecular genetic breeding, in particular to two PARMS-SNP (Penta-primer amplification refractory mutation system-single nucleotide polymorphism) molecular markers for identifying a resistant gene VrTAF5 of Vigna radiata (Linn.) Wilczek Cercospora leaf spot disease (CLS).

### BACKGROUND

Vigna radiata (Linn.) Wilczek leaf spot disease is the main fungal disease in the growth of Vigna radiata (Linn.) Wilczek, which occurs in China and other Vigna radiata (Linn.) Wilczek producing area in Asia. The disease is caused by several species of Cercospora, among which Cercospora canescens Ellis and Martin is the main pathogen, which mainly infects the leaves of plants, and suffers the most in flowering and pod bearing period. Selecting resistant varieties can effectively reduce the occurrence of diseases in Vigna radiata (Linn.) Wilczek planting. Although many germplasm resources of Vigna radiata (Linn.) Wilczek resistant to leaf spot disease have been found, however, only one study has reported QTL-qCLS, which can explain 65.5-80.5% of phenotypic variation of resistance.

Phenotypic identification of Vigna radiata (Linn.) Wilczek leaf spot disease costs lots of time and laborious, and identification in field is easily affected by environment. Therefore, compared with the phenotypic identification, molecular markers relate to disease resistance traits have the advantages of accuracy, economy and rapidity. Functional molecular markers are molecular markers developed according to polymorphic sequences within functional genes closely relate to phenotypic traits with advantages that they can accurately and reliably lock target genes, accurately respond to genetic variations of functional alleles, and have more reliable genetic effects. Single nucleotide polymorphism (SNP) is a polymorphism produced by single nucleotide variation, so it is difficult to distinguish its polymorphism by using the difference in length between conventional Polymerase chain reaction (PCR) and gel electrophoresis. Penta-primer amplification refractory mutation system (PARMS) is a new SNP typing technology, which has lower cost than imported detection reagent Kompetitive Allele Specific PCR (KASP), and when DNA is alkaline cracked, the PARMS is more densely clustered than the KASP.

### SUMMARY

In order to solve the problems existing in the prior art, an objective of the invention is to provide PARMS-SNP molecular markers for identifying resistant gene VrTAF5 of Vigna radiata (Linn.) Wilczek Cercospora leaf spot disease and a primer thereof, which can quickly identify Vigna radiata (Linn.) Wilczek resistant varieties, and meet needs of assisting Vigna radiata (Linn.) Wilczek molecular breeding and shortening a breeding time.

To realize the above objective, in an aspect, the invention provides two PARMS-SNP molecular markers for identifying the resistant gene VrTAF5 of Vigna radiata (Linn.) Wilczek Cercospora leaf spot disease, which include SNP sites PARMS-1517 and PARMS-10010; a 240 bp sequence from before to after the SNP site PARMS-1517 is shown in SEQ ID NO.1, a 131st position is the SNP site PARMS-1517, and a polymorphism is A/C; a 240 bp sequence from before to after the SNP site PARMS-10010 is shown in SEQ ID NO.2, a 117th position is the SNP site PARMS-10010, and a polymorphism is A/G.

In another aspect, the invention further provides a primer set for identifying the two PARMS-SNP molecular markers, and the primer set includes a PARMS-1517 primer set and a PARMS-10010 primer set. The PARMS-1517 primer set includes primers PARMS- 1517F1, PARMS-1517F2 and PARMS-1517R: nucleotide sequences of PARMS-1517F1 are shown in SEQ ID NO.3, that of PARMS-1517F2 are shown in SEQ ID NO.4, and that of PARMS-1517R are shown in SEQ ID NO.5. The PARMS-10010 primer set includes primers PARMS-10010F1, PARMS-10010F2 and PARMS-10010R: nucleotide sequences of PARMS-10010F1 are as shown in SEQ ID NO.6, that of PARMS-10010F2 are as shown in SEQ ID NO.7, and that of PARMS-10010R are as shown in SEQ ID NO.8.

In still another aspect, the invention further provides an application of the two PARMS-SNP molecular markers or the primer set in assisting Vigna radiata (Linn.) Wilczek breeding.

In even another aspect, the invention further provides an application of the two PARMS-SNP molecular markers in identifying the resistant gene VrTAF5 of Vigna radiata (Linn.) Wilczek Cercospora leaf spot disease.

In further another aspect, the invention further provides an application method of the primer set, the primer set is applied to identify the resistant gene VrTAF5 of Vigna radiata (Linn.) Wilczek Cercospora leaf spot disease.

In an embodiment of the invention, specific steps for identifying the resistant gene VrTAF5 of Vigna radiata (Linn.) Wilczek Cercospora leaf spot disease are as follows: performing a PCR amplification on Vigna radiata (Linn.) Wilczek genome DNA by using the primer set; reading fluorescent signals after the PCR amplification, analyzing and converting the fluorescent signals, and identifying the analyzed and converted fluorescent signals to obtain genotypes belonging to susceptible homozygous AA type, resistant homozygous CC or GG type, and heterozygous CA or AG type.

In an embodiment of the invention, a PCR amplification reaction system of the PARMS-1517 primer set contains 1 μL of template DNA, 5 μL of 2×PARMS master mix, 0.15 μL of primer PARMS-1517F1 in 10 μM, 0.15 μL of primer PARMS-1517F2 in 10 μM, 0.4 μL of primer PARMS-1517R in 10 μM and 3.3 μL of ddH₂O.

A PCR amplification reaction system of PARMS-10010 primer set contains 1 μL of template DNA, 5 μL of 2×PARMS master mix, 0.15 μL of primer PARMS-10010F1 in 10 μM, 0.15 μL of primer PARMS-10010F2 in 10 μM, 0.4 μL of primer PARMS-10010R in 10 μM and 3.3 μL of ddH₂O.

In an embodiment of the invention, procedures of the PCR amplification are as follow: thermal activating at 94° C. for 15 min, denaturing at 94° C. for 20 s, annealing and extension at 57-65° C. for 1 min, 10 cycles; denaturing at 94° C. for 20 s, annealing and extending at 57° C. for 1 min, 30 cycles.

In an embodiment of the invention, the fluorescence signals are read by TECAN infinite M1000 microplate reader. On-line software SNP Decoder (http://www.snpway.com/snpdecoder/) is used to analyze and convert the fluorescence signals.

In even further another aspect, the invention further provides a Vigna radiata (Linn.) Wilczek Cercospora leaf spot disease resistance detection kit, which includes the primer set for identifying the two PARMS-SNP molecule makers.

The invention discloses the following technical effects: the invention provides two PARMS-SNP molecular markers for identifying a resistant gene VrTAF5 of Vigna radiata (Linn.) Wilczek Cercospora leaf spot disease and a primer set for identifying the two PARMS-SNP molecular markers, which have the characteristics of specificity, accuracy, reliability, simple operation so as to quickly identify Vigna radiata (Linn.) Wilczek resistant varieties, meet the needs of assisting Vigna radiata (Linn.) Wilczek molecular breeding and shorten a breeding period.

### BRIEF DESCRIPTION OF DRAWINGS

In order to explain the embodiments of the invention or the technical solution in the prior art more clearly, the following will briefly introduce the drawings used in the embodiments. Obviously, the drawings in the following description are only some embodiments of the invention, and for those skilled in the art, other drawings can be obtained according to these drawings without paying creative labor.

FIG. 1 is a comparative picture of proteins encoded by resistant gene VrTAF5 of Vigna radiata (Linn.) Wilczek Cercospora leaf spot disease of resistant parent V4718 and susceptible parent KPS1.

FIG. 2A is PARMS-1517 typing diagram of 186 samples to be tested.

FIG. 2B is PARMS-10010 typing diagram of 186 samples to be tested.

### DETAILED DESCRIPTION OF EMBODIMENTS

The following will clearly and completely describe the technical solution in the embodiments of the invention with reference to the drawings in the embodiments of the invention. Obviously, the described embodiments are only part of the embodiments of the invention, not all of them. Based on the embodiments of the invention, all other embodiments obtained by those skilled in the art without creative labor belong to the scope of protection of the invention.

In order to make the above objectives, features and advantages of the invention clearer and easier to understand, the invention will be further explained in detail with reference to the drawings and illustrated embodiments.

### Embodiment 1

### Design PARMS-SNP Molecular Marker of Resistant Gene VrTAF5 of Vigna radiata (Linn.) Wilczek Cercospora Leaf Spot Disease

The major QTL-qCLS has been finely located in genomic region of −13 kilobases (Kb) on chromosome 6 by using BC₈F₂ and F2 populations of Vigna radiata (Linn.) Wilczek Cercospora leaf spot disease resistant variety V4718 and susceptible variety KPS1. The genomic region contains only one annotated gene LOC106765332 (named “VrTAF5”) encoding TATA binding protein related factor 5(TAFS), which is a subunit of TFIID and SAGA complex.

### Sequencing the Sequences of Resistant Gene VrTAF5 in the Susceptible Variety KPS1 and the Resistant Variety V4718

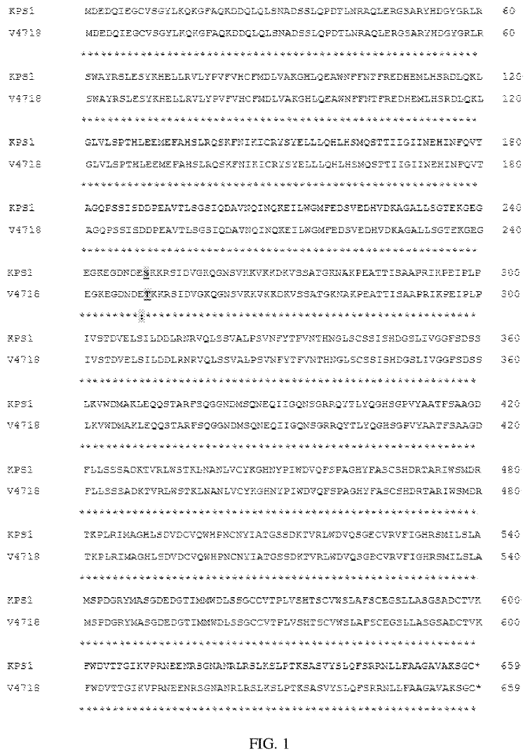

Comparing the sequences of the resistant gene VrTAF5 between the two parents mentioned above, it shows multiple single nucleotide polymorphisms (SNPs) and insertions/deletions (InDels), among which 8 SNPs exist in 8 different exons, while one SNP(G4,932C) exists in exon 8, which causes amino acid mutation (S250T). The mutation results are shown in FIG. 1, and the mutation sites are marked by horizontal lines in FIG. 1.

Seven PARMS-SNP molecular markers: PARMS-1517, PARMS-1739, PARMS-4932, PARMS-9548, PARMS-9884, PARMS-10010 and PARMS-10228 are designed and developed. According to the seven single nucleotide differences of gene sequences between the V4718 and the KPS1, PARMS primer sets containing seven SNP differences are designed by Primer5.0, and each primer set contains two specific primers and one universal primer.

According to the invention, PARMS-SNP detection technology and a high-throughput SNP detection platform GeneMatrix are adopted for sample analysis and detection.

According to the results of resistance evaluation, the PARMS-1517 and the PARMS-10010 have a higher screening rate of molecular markers. The PARMS-1517 is based on the nucleotide difference at position 32622352 on chromosome 6 of Vigna radiata (Linn.) Wilczek. The sequence of 240 base pairs (bp) before and after the PARMS-1517 is shown in Sequence (SEQ) ID NO.1, and the 131st position is the PARMS-1517, with polymorphism of Adenine/Cytosine (A/C). The PARMS-10010 is based on the nucleotide difference at position 32613913 on chromosome 6 of Vigna radiata (Linn.) Wilczek. The sequence of 240 bp before and after the PARMS-10010 is shown in SEQ ID NO.2, and the 117th position is the PARMS-10010 with polymorphism of Adenine/Guanine (A/G), which can be used to identify the resistance of Vigna radiata (Linn.) Wilczek varieties to Cercospora leaf spot disease.

Primer sequences of PARMS-1517 molecular markers are shown as follows:

Primer 1: PARMS-1517F1:
5′ GAAGGTGACCAAGTTCATGCTGTTCTGCTAGATACCACGATGGA 3′
Primer 2: PARMS-1517F2:
5′ GAAGGTCGGAGTCAACGGATTTTCTGCTAGATACCACGATGGC 3′
Primer 3: PARMS-1517R:
5′ GACCTATATGCCCATGATCTCAG 3′.

Primer sequences of PARMS-10010 molecular markers are shown as follows:

Primer 1: PARMS-10010F1:
5′GAAGGTGACCAAGTTCATGCTTTTAATGATCTGAGTCTGTTAGCAT
TT 3′
Primer 2: PARMS-10010F2:
5′GAAGGTCGGAGTCAACGGATTTTTAATGATCTGAGTCTGTTAGCAT
TC 3′
Primer 3: PARMS-10010R:
5′TCTTTAGGAATTTTCTTTTCTAATGG 3′.

### Embodiment 2

### Application of PARMS-SNP Markers of Vigna radiata (Linn.) Wilczek Cercospora Leaf Spot Disease Resistant Gene in Screening Vigna radiata (Linn.) Wilczek Resistant Varieties

Genome DNA of 186 Vigna radiata (Linn.) Wilczek varieties to be detected (as shown in Table 1) is extracted, and each DNA concentration is determined by spectrophotometer. Take that DNA of Vigna radiata (Linn.) Wilczek variety to be detected as a template, and carry out polymerase chain reaction (PCR) amplification by using the primer set, namely PARMS reaction detection.

PCR system (10 microliters (μL)): 5 μL of 2×PARMS master mix, 0.15 μL of primer 1 (10 micromoles per liter (μM)) ,0.15 μL of primer 2 (10 μM), 0.4 μL of primer 3 (10 μM), 1 μL of template DNA (50 ng/μL), 3.3 μL of double distilled H₂O (ddH₂O).

PCR reaction procedures: heat activating at 94° C. for 15 minutes (min); denaturing at 94° C. for 20 seconds (s), annealing at 57-65° C., extending for 1 min, 10 cycles; denaturing at 94° C. for 20 s, annealing and extending at 57° C. for 1 min, 30 cycles.

After PCR, the fluorescence signals are read by TECAN infinite M1000 microplate reader. The fluorescence signals are further analysed and converted by online software SNP Decoder (http://www.snpway.com/snpdecoder/), so that a clear and intuitive typing map is obtained, and the genotype results are output according to different colors.

If only base A is detected at SNP site PARMS-1517, it is identified that the Vigna radiata (Linn.) Wilczek sample to be detected has a susceptible homozygous genotype; if only base C is detected, the Vigna radiata (Linn.) Wilczek sample is detected to have a resistant homozygous genotype; if both base A and base C are detected, it is determined that the Vigna radiata (Linn.) Wilczek sample to be detected has a heterozygous genotype.

If only base A is detected at SNP site PARMS-10010, it is identified that the Vigna radiata (Linn.) Wilczek sample to be detected has a susceptible homozygous genotype; if only base G is detected, the Vigna radiata (Linn.) Wilczek sample to be detected is identified to have a resistant homozygous genotype; if both base A and base G are detected, it is judged that the Vigna radiata (Linn.) Wilczek sample to be detected has a heterozygous genotype.

The indoor inoculation resistance of Vigna radiata (Linn.) Wilczek varieties is identified, and the specific steps are as follows:

the resistance of Vigna radiata (Linn.) Wilczek is evaluated by indoor artificial inoculation, and healthy Vigna radiata (Linn.) Wilczek plants are inoculated at the leaf-regrowth stage, which is kept in a moisture shed for 36 hours and then grew normally at 25° C.

The incidence of Cercospora leaf spot disease is observed 15 days after inoculation.

Evaluation of resistance refers to Disease Classification Standard (Wu Quan' an, 1991):

Grade 0: no visible infection on leaves;

Grade 1: there are only small disease spots on the leaves, accounting for less than 2% of the leaf area;

Grade 3: the spots are small, with a diameter of 1-2 millimeters (mm) and no halo of chlorosis, accounting for 3%-25% of the leaf area;

Grade 5: the spot has a larger diameter of 2.1-5 mm, and has chlorotic halo, accounting for 26%-50% of the leaf area;

Grade 7: the disease spots are large, the diameter is more than 5.1 mm accounting for 51%-75% of the leaf area, and some leaves are dead;

Grade 9: the disease spots are connected into a piece, accounting for more than 76% of the leaf area, with a large number of sporulation and serious deciduous leaves.

Evaluation criteria of resistance (classified by disease index):

resistance-(R): disease index of 0-20.0; middle resistance (MR): disease index of 20.0-40.0; middle susceptibility (MS): disease index of 40.1-60.0; susceptibility (S) disease index of 60.1-80.0; high susceptibility (HS): disease index of 80.1-100.

Disease index=(number of disease plants at all levels×disease grade value)/(total number of investigated strains×highest level value) ×100 Calculation of disease index:

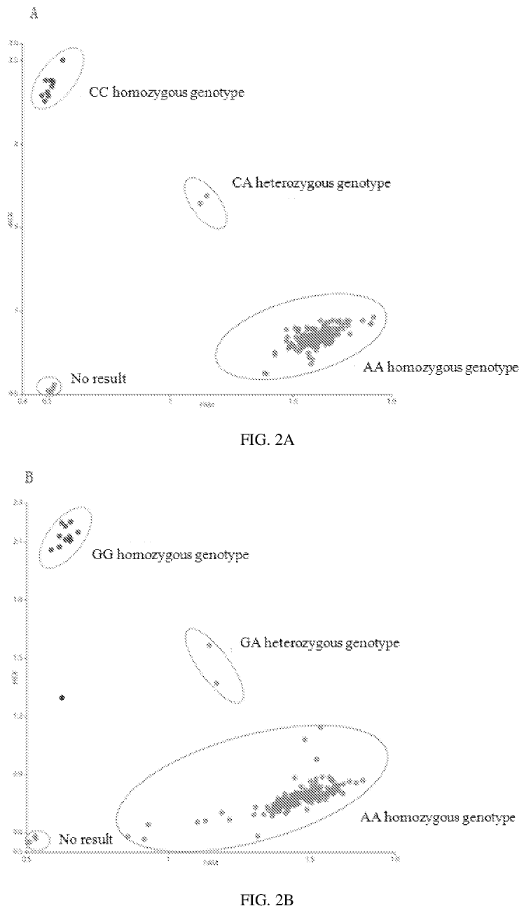

The results of C/A genotype and disease index are shown in Table 1, and the genotyping results are shown in FIG. 2A. There are 12 genotypes of CC and CA, among which 9 are resistant varieties, accounting for 75%. A total of 174 AA genotypes are detected, of which 141 are susceptible, accounting for 81.03%.

The corresponding results of G/A genotype and disease index are shown in Table 1, and the genotyping results are shown in FIG. 2B. There are 12 genotypes of GG and GA, among which 9 are resistant varieties, accounting for 75%. A total of 174 AA genotypes are detected, of which 141 are susceptible, accounting for 81.03%.

TABLE 1
Corresponding results of genotype and disease index

| | | Resistance | PARMS- | PARMS- |
|---|---|---|---|---|
| NO. | Variety | evaluation | 1517 | 10010 |
| 1 | Xinyu | HS | AA | AA |
| | L13012 | | | |
| 2 | Xinyu | S | AA | AA |
| | L13013 | | | |
| 3 | Xinyu | S | AA | AA |
| | L13014 | | | |
| 4 | Xinyu | S | AA | AA |
| | L13015 | | | |
| 5 | Xinyu | MS | AA | AA |
| | L13016 | | | |
| 6 | Xinyu | S | AA | AA |
| | L13017 | | | |
| 7 | Xinyu | MS | AA | AA |
| | L13018 | | | |
| 8 | Xinyu | S | AA | AA |
| | L13019 | | | |
| 9 | Xinyu | MS | AA | AA |
| | L13020 | | | |
| 10 | Xinyu | S | AA | AA |
| | L13021 | | | |
| 11 | Xinyu | R | AA | AA |
| | L13022 | | | |
| 12 | Xinyu | MR | AA | AA |
| | L13023 | | | |
| 13 | L67-1 | S | AA | AA |
| 14 | AL68 | S | AA | AA |
| 15 | AL81 | S | AA | AA |
| 16 | Sanxia | MS | AA | AA |
| | NO. 4 | | | |
| 17 | Sanxia | HS | AA | AA |
| | NO. 5 | | | |
| 18 | Mizhi Vigna | MS | AA | AA |
| | radiata | | | |
| | (Linn.) | | | |
| | Wilczek | | | |
| 19 | NO. 6 Mizhi | R | CC | GG |
| | Vigna | | | |
| | radiata | | | |
| | (Linn.) | | | |
| | Wilczek | | | |
| 20 | Yanglinghei | R | AA | AA |
| | Vigna | | | |
| | radiata | | | |
| | (Linn.) | | | |
| | Wilczek | | | |
| 21 | Yan'an | MS | AA | AA |
| | NO. 4 | | | |
| 22 | Sanxia | MS | AA | AA |
| | NO. 2 | | | |
| 23 | Sulyu NO. 1 | MS | AA | AA |
| 24 | Sulyu NO. 4 | HS | AA | AA |
| 25 | Sulyu NO. 5 | MS | AA | AA |
| 26 | Sulyu NO. 6 | MS | AA | AA |
| 27 | Sulyu NO. 7 | MR | CC | GG |
| 28 | Yan'an | HS | AA | AA |
| | Vigna | | | |
| | radiata | | | |
| | (Linn.) | | | |
| | Wilczek | | | |
| 29 | Danlyu | HS | AA | AA |
| | NO. 1 | | | |
| 30 | Kanglyu | MR | AA | AA |
| | NO. 4 | | | |
| 31 | Zhonglyu | HS | AA | AA |
| | NO. 3 | | | |
| 32 | Jilyu NO. 2 | MS | AA | AA |
| 33 | Wild | HS | AA | AA |
| | variety 1 | | | |
| 34 | Wild | MR | AA | AA |
| | variety 2 | | | |
| 35 | Wild | MS | AA | AA |
| | variety 3 | | | |
| 36 | Wild | MS | AA | AA |
| | variety 4 | | | |
| 38 | Wild | S | AA | AA |
| | variety 6 | | | |
| 39 | Wild | MR | AA | AA |
| | variety 7 | | | |
| 40 | Wild | S | AA | AA |
| | variety 8 | | | |
| 41 | V1197 | HS | AA | AA |
| 42 | Wild | S | AA | AA |
| | variety 10 | | | |
| 43 | V4718 | R | CC | GG |
| 44 | RUM5 | S | AA | AA |
| 45 | KPS2 | HS | AA | AA |
| 46 | CN84-1 | MR | AA | AA |
| 47 | JP229096 | MR | AA | AA |
| 48 | VC1560A | MR | AA | AA |
| 49 | JP229175 | MS | AA | AA |
| 50 | V2533 | MS | AA | AA |
| 51 | V1067AG | MS | AA | AA |
| 52 | Zhenglyu | S | AA | AA |
| | NO. 9 | | | |
| 53 | JP229241 | MR | CC | GG |
| 54 | JP229121 | MR | AA | AA |
| 55 | V1945AG | MS | AA | AA |
| 56 | V1616AG | S | AA | AA |
| 57 | Tao 98502 | MS | AA | AA |
| 58 | JP231233 | MR | AACC | AAGG |
| 59 | Pinlyuyouzi | S | AA | AA |
| 60 | B23 | MR | AA | AA |
| 61 | V2808BG | S | AA | AA |
| 62 | JP240329 | MS | AA | AA |
| 63 | JP226698 | HS | AA | AA |
| 64 | JP240379 | S | AA | AA |
| 65 | JP78926 | S | AA | AA |
| 66 | Bailyu | MR | AA | AA |
| | NO. 8 | | | |
| 67 | RG066 | S | AA | AA |
| 68 | V2481AG | MS | AA | AA |
| 69 | B30 | S | AA | AA |
| 70 | C2969 | S | AA | AA |
| 72 | V2191BG | S | AA | AA |
| 74 | Gaoyang | HS | AA | AA |
| | Vigna | | | |
| | radiata | | | |
| | (Linn.) | | | |
| | Wilczek | | | |
| 75 | B37 | MR | AA | AA |
| 76 | Yulyu NO. 2 | MR | CC | GG |
| 77 | JP78938 | S | AA | AA |
| 78 | Liaolyu | S | AA | AA |
| | NO. 8 | | | |
| 79 | V2984BG | S | AA | AA |
| 80 | V1586BG | S | AA | AA |
| 81 | JP229403 | MS | AA | AA |
| 82 | Zhonglyu | MS | AA | AA |
| | NO. 8 | | | |
| 83 | Lyufeng | HS | AA | AA |
| | NO. 2 | | | |
| 84 | JP229254 | S | AA | AA |
| 85 | V1394AG | S | AA | AA |
| 86 | JP78938 | S | AA | AA |
| 87 | B49 | S | AA | AA |
| 88 | V1377AG | MS | AA | AA |
| 89 | P22008 | MR | AA | AA |
| 90 | V2272AG | MR | CC | GG |
| 91 | JP202271 | HS | AA | AA |
| 92 | V2709BG | S | AA | AA |
| 93 | B55 | S | AA | AA |
| 94 | JP229130 | MS | AA | AA |
| 95 | JP240384 | MS | AA | AA |
| 96 | Zhonglyu | MR | AA | AA |
| | NO. 11 | | | |
| 97 | JP240338 | MR | AA | AA |
| 98 | Tonglyu | S | AA | AA |
| | 918 | | | |
| 99 | JP229193 | MS | AA | AA |
| 100 | JP229216 | S | AA | AA |
| 101 | V1188 | MS | AA | AA |
| 102 | V22T8AG | MR | AA | AA |
| 103 | KPS1 | HS | AA | AA |
| 104 | B69 | S | AA | AA |
| 105 | 22172 | S | AA | AA |
| 106 | JP229190 | MS | CC | GG |
| 107 | V1387AG | MR | AA | AA |
| 108 | V2066BG | MS | AA | AA |
| 109 | V2419 | MR | AA | AA |
| 110 | P24029 | S | AA | AA |
| 111 | V1948AG | S | AA | AA |
| 113 | AL009 | HS | AA | AA |
| 114 | AL010 | MR | AA | AA |
| 115 | AL028 | S | AA | AA |
| 116 | IS0028 | MR | AA | AA |
| 117 | AL153 | MR | AA | AA |
| 118 | AL155*1 | S | AA | AA |
| 119 | AL152*1 | S | AA | AA |
| 120 | LD368 | MR | AA | AA |
| 121 | L158 | MS | AA | AA |
| 122 | AL090*1 | MR | AA | AA |
| 123 | AL152*2 | MS | AA | AA |
| 124 | AL160 | S | AA | AA |
| 125 | AL156 | MS | CC | GG |
| 126 | AL154 | MR | AA | AA |
| 127 | AL158*1 | S | AA | AA |
| 128 | IS0018 | S | AA | AA |
| 129 | AL084*2 | S | AA | AA |
| 130 | CO1522 | MS | AA | AA |
| 131 | CO1595 | HS | AA | AA |
| 132 | COOO1718 | S | AA | AA |
| 133 | AL084*1 | S | AA | AA |
| 134 | COOO1724 | HS | AA | AA |
| 135 | COOO1725 | S | AA | AA |
| 136 | COOO1726 | HS | AA | AA |
| 137 | COOO2976 | S | AA | AA |
| 138 | COOO2979 | S | AA | AA |
| 139 | A17 | HS | AA | AA |
| 140 | COOO2982 | MR | AA | AA |
| 141 | COOO3403 | HS | AA | AA |
| 142 | COOO4252 | S | AA | AA |
| 143 | 23403 | S | AA | AA |
| 144 | L151 | MS | AA | AA |
| 145 | AL085 | MS | AA | AA |
| 146 | JP240383 | S | AA | AA |
| 147 | V1301BG | MS | AA | AA |
| 148 | JP31224 | S | AA | AA |
| 149 | JP107875 | MR | AACC | AAGG |
| 150 | V2013BG | MS | AA | AA |
| 151 | A44 | S | AA | AA |
| 152 | A45 | MS | AA | AA |
| 153 | A46 | S | AA | AA |
| 154 | JP229185 | S | AA | AA |
| 155 | Tao 9806 | HS | AA | AA |
| 156 | Jilyu NO. 5 | S | AA | AA |
| 157 | Nanyang | MR | AA | AA |
| | Vigna | | | |
| | radiata | | | |
| | (Linn.) | | | |
| | Wilczek | | | |
| 158 | Bailyu | MR | AA | AA |
| | NO. 9 | | | |
| 159 | Zhonglyu | MS | AA | AA |
| | NO. 5 | | | |
| 160 | Huailyu | MS | AA | AA |
| | NO. 8 | | | |
| 161 | Jinlyu NO. 8 | MS | AA | AA |
| 162 | Inner | MR | AA | AA |
| | Mongolia | | | |
| | Vigna | | | |
| | radiata | | | |
| | (Linn.) | | | |
| | Wilczek | | | |
| 163 | Zhonglyu | MS | AA | AA |
| | NO. 15 | | | |
| 164 | A57 | HS | AA | AA |
| 165 | Zhonglyu | MS | AA | AA |
| | NO. 4 | | | |
| 166 | Yulyu NO. 1 | MR | AA | AA |
| 167 | A18 | S | AA | AA |
| 168 | V2010BG | MR | AA | AA |
| 169 | Liaolyu | MS | AA | AA |
| | NO.3 | | | |
| 170 | V2036AG | MS | AA | AA |
| 171 | JP240386 | MS | CC | GG |
| 172 | JP231216 | MS | AA | AA |
| 173 | JP229144 | MR | AA | AA |
| 174 | JP240327 | S | AA | AA |
| 175 | JP240342 | HS | AA | AA |
| 176 | Oil Vigna | MS | AA | AA |
| | radiata | | | |
| | (Linn.) | | | |
| | Wilczek | | | |
| 177 | V1944BY | S | AA | AA |
| 178 | V1411AG | S | AA | AA |
| 179 | Baolyu 942 | MS | AA | AA |
| 180 | V1953bg | MS | AA | AA |
| 181 | Dayang | MS | AA | AA |
| | Vigna | | | |
| | radiata | | | |
| | (Linn.) | | | |
| | Wilczek | | | |
| 182 | JP99006 | S | AA | AA |
| 183 | V2007BG | MS | AA | AA |
| 184 | V3726BG | R | CC | GG |
| 185 | P81015 | S | AA | AA |
| 186 | V1476AG | MS | AA | AA |
| 187 | JP240343 | MS | AA | AA |
| 188 | JP98811 | MS | AA | AA |
| 189 | A82 | MR | AA | AA |
| 190 | V4908AB | S | AA | AA |

It can be seen from the Table 1 that the PARMS-SNP markers for identifying the resistant gene of Vigna radiata (Linn.) Wilczek Cercospora leaf spot disease disclosed by the invention has the characteristics of specificity, accuracy, reliability, simple operation and the like, and can quickly identify resistant varieties, assist molecular breeding and shorten a breeding period.

The above embodiments only describe the preferred mode of the invention, but do not limit the scope of the invention. On the premise of not departing from the design spirit of the invention, various modifications and changes made by those skilled in the field to the technical solution of the invention shall fall within the protection scope determined by the claims of the invention.

What is claimed is:

1. An application method of a primer set of two penta-primer amplification refractory mutation system-single nucleotide polymorphism (PARMS-SNP) molecular markers for identifying a resistant gene VrTAF5 of Vigna radiata (Linn.) Wilczek Cercospora leaf spot disease, wherein the primer set of the two PARMS-SNP molecular markers is applied to assist in screening of Vigna radiata (Linn.) Wilczek Cercospora leaf spot disease resistant varieties,
wherein the two PARMS-SNP molecular markers comprise SNP sites PARMS-1517 and PARMS-10010;
wherein a 240 base pairs (bp) sequence from before to after the SNP site PARMS-1517 is shown in Sequence (SEQ) ID NO.1, a 131st position is the SNP site PARMS-1517, and a polymorphism is Adenine/Cytosine (A/C); a 240 bp sequence from before to after the SNP site PARMS-10010 is shown in SEQ ID NO.2, a 117th position is the SNP site PARMS-10010, and a polymorphism is Adenine/Guanine (A/G);
wherein the primer set comprises a PARMS-1517 primer set and a PARMS-10010 primer set;
wherein PARMS-1517 primer set comprises primers PARMS-1517F1, PARMS-1517F2 and PARMS-1517R: a nucleotide sequence of PARMS-1517F1 is shown in SEQ ID NO.3, a nucleotide sequence of PARMS-1517F2 is shown in SEQ ID NO.4, and a nucleotide sequence of PARMS-1517R is shown in SEQ ID NO.5; and
wherein the PARMS-10010 primer set comprises primers PARMS-10010F1, PARMS-10010F2 and PARMS-10010R: a nucleotide sequence of PARMS-10010F1 is as shown in SEQ ID NO.6, a nucleotide sequence of PARMS-10010F2 is as shown in SEQ ID NO.7, and a nucleotide sequence of PARMS-10010R is as shown in SEQ ID NO.8.

2. An application method of a primer set of two PARMS-SNP molecular markers, wherein the primer set of the two PARMS-SNP molecular markers is applied to identify a resistant gene VrTAF5 of Vigna radiata (Linn.) Wilczek Cercospora leaf spot disease,
wherein the two PARMS-SNP molecular markers comprise SNP sites PARMS-1517 and PARMS-10010;
wherein a 240 base pairs (bp) sequence from before to after the SNP site PARMS-1517 is shown in SEQ ID NO.1, a 131st position is the SNP site PARMS-1517, and a polymorphism is A/C; a 240 bp sequence from before to after the SNP site PARMS-10010 is shown in SEQ ID NO.2, a 117th position is the SNP site PARMS-10010, and a polymorphism is A/G;
wherein the primer set comprises a PARMS-1517 primer set and a PARMS-10010 primer set;
wherein PARMS-1517 primer set comprises primers PARMS-1517F1, PARMS-1517F2 and PARMS-1517R: a nucleotide sequence of PARMS-1517F1 is shown in SEQ ID NO.3, a nucleotide sequence of PARMS-1517F2 is shown in SEQ ID NO.4, and a nucleotide sequence of PARMS-1517R is shown in SEQ ID NO.5; and
wherein the PARMS-10010 primer set comprises primers PARMS-10010F1, PARMS-10010F2 and PARMS-10010R: a nucleotide sequence of PARMS-10010F1 is as shown in SEQ ID NO.6, a nucleotide sequence of PARMS-10010F2 is as shown in SEQ ID NO.7, and a nucleotide sequence of PARMS-10010R is as shown in SEQ ID NO.8.

3. The application method according to claim 2, wherein specific steps for identifying the resistant gene VrTAF5 of Vigna radiata (Linn.) Wilczek Cercospora leaf spot disease comprises:
performing a polymerase chain reaction (PCR) amplification on Vigna radiata (Linn.) Wilczek genome DNA by using the primer set;
reading fluorescent signals after the PCR amplification;
analyzing and converting the fluorescent signals; and
identifying the analyzed and converted fluorescent signals to obtain genotypes belonging to susceptible homozygous AA type, resistant homozygous CC or GG type, and heterozygous CA or AG type.

4. The application method according to claim 3, wherein a PCR amplification reaction system of the PARMS-1517 primer set contains 1 microliter (μL) of template DNA, 5 μL of 2×PARMS master mix, 0.15 μL of primer PARMS-1517F1 in 10 micromoles per liter (μM), 0.15 μL of primer PARMS-1517F2 in 10 μM, 0.4 μL of primer PARMS-1517R in 10 μM and 3.3 μL of double distilled H₂O (ddH₂O);
a PCR amplification reaction system of the PARMS-10010 primer set contains 1 μL of template DNA, 5 μL of 2×PARMS master mix, 0.15 μL of primer PARMS-10010F1 in 10 μM, 0.15 μL of primer PARMS-10010F2 in 10 μM, 0.4 μL of primer PARMS-10010R in 10 μM and 3.3 μL of ddH₂O.

5. The application method according to claim 3, wherein procedures of the PCR amplification are as follow:
thermal activating at 94° C. for 15 minutes (min), denaturing at 94° C. for 20 seconds (s), annealing and extension at 57-65° C. for 1 min, 10 cycles;
denaturing at 94° C. for 20 s, annealing and extending at 57° C. for 1 min, 30 cycles.

6. The application method according to claim 3, wherein the fluorescence signals are read by TECAN infinite M1000 microplate reader; on-line software snpdecoder is used to analyze and convert the fluorescence signals.
